(19) United States
(12) Patent Application Publication
(10) Pub. No.: US 2026/0266838 A1
(43) Pub. Date: Sep. 10, 2026

(54) CC16-HNE Assay

(71) Applicant: Nordic Bioscience A/S, Herlev (DK)

(72) Inventors: Jannie Marie Bülow Sand, Målov (DK); Annika Hummersgaard Hansen, København Ø (DK); Diana Julie Leeming, Klampenborg (DK); Morten Asser Karsdal, Kobenhavn Ø (DK)

(73) Assignee: Nordic Bioscience A/S, Herlev (DK)

(21) Appl. No.: 19/165,281

(22) PCT Filed: Mar. 18, 2024
(86) PCT No.: PCT/EP2024/057165

§ 371 Date: Sep. 15, 2025

(30) Foreign Application Priority Data
Mar. 16, 2023 (GB) 2303879.7

(51) Int. Cl. G01N 33/68; C07K 16/18

(52) U.S. Cl. CPC G01N 33/6884; C07K 16/18; G01N 2470/10; G01N 2800/12; G01N 2800/122

(57) ABSTRACT

Disclosed herein are methods of immunoassay for detecting HNE-generated fragments of club cell secretory protein 16 (CC16) in a patient sample, which methods can be used for detecting and/or monitoring chronic obstructive pulmonary disease (COPD) or idiopathic pulmonary fibrosis (IPF) or a particular level of severity thereof in a patient. Also disclosed are monoclonal antibodies and assay kits for use in said methods of immunoassay.

### FIELD OF THE INVENTION

The present invention relates to methods of immunoassay for detecting HNE-generated fragments of club cell secretory protein 16 (CC16) in a patient sample, and the use thereof for detecting and/or monitoring chronic obstructive pulmonary disease (COPD) or idiopathic pulmonary fibrosis (IPF) or a particular level of severity thereof in a patient. The present invention also relates to monoclonal antibodies and assay kits for use in said methods of immunoassay.

### BACKGROUND

Chronic pulmonary diseases are a common and a significant health concern globally [1]. Chronic obstructive pulmonary disease (COPD) is a prevalent pulmonary disease and has been estimated to affect 212.3 million people worldwide in 2019 [2]. In comparison, idiopathic pulmonary fibrosis (IPF) is considered a rare disease, estimated to affect 0.3-4.51 per 10,000 globally as of 2021, and with a detrimental effect on health-related quality of life [3]. The pathological mechanisms driving both diseases are inherently different but are believed to share degrees of overlap [4]. Overall, IPF is characterized by scarring of the lung with increased deposition of extracellular matrix (ECM) proteins, in contrast to COPD that is mainly characterized by derailed inflammation and loss of tissue integrity due to increased degradation of the ECM.

As noted, IPF is characterized by lung tissue scarring, epithelial damage and pathological change of airway morphology [19]. Pulmonary hypertension (PH) is a comorbidity affecting IPF patients and is categorized as part of PH group 3 [20]. IPF-PH is associated with a higher risk of mortality, and development of new therapies is difficult due to current limitations of disease knowledge [20]. Additionally, diagnosing PH is challenging since the gold-standard is the invasive procedure of right heart catheterization [21].

Club cells are largely present in the epithelium of the lower airways, secreting surfactant and various other proteins [5], [6]. Club cells are the main producers of club cell secretory protein 16 (CC16), also known as CC10, secretoglobin, or uteroglobin. The exact biological functions of CC16 have yet to be fully elucidated but it is believed to have anti-inflammatory properties, protecting the airways from inflammation and oxidative stress [7]. CC16 has been proposed as a biomarker for lung epithelial injury, where increased circulating levels are believed to reflect club cell damage and/or increased epithelial permeability.

Studies in mice have shown that CC16 knock-out significantly increased the induction of ECM remodeling and inflammation following cigarette smoke exposure [8]. Secretion of CC16 has been shown to be elevated in serum and bronchoalveolar lavage fluid in IPF [9]. In contrast, CC16 levels were decreased in serum from COPD patients when compared to non-smoking controls and lower levels were associated with more severe disease [10], [11]. This is in line with a study showing the highest degree of CC16 staining in the airway epithelium from healthy non-smokers, a reduction in healthy smokers and low/absent expression in COPD tissue [8]. Additionally, the severity of COPD was suggested to alter the expression of CC16, showing a lower degree of CC16 staining in severe compared to mild COPD as determined by the Global Initiative for Obstructive Lung Disease (GOLD) stage [8]. Interestingly, one study showed that low levels of serum CC16 was related to accelerated lung function decline (FEV1) [12]. This suggests opposing effects of CC16 in COPD and IPF patients which may contribute to COPD being more associated with inflammation than IPF. A clinical trial that combined immunosuppressive and anti-inflammatory agents for IPF showed a harming effect on the patients, which terminated the trial prematurely due to increased rates of death and hospitalizations [13]. Thus, some degree of inflammation might be serving a protective purpose in IPF.

### SUMMARY OF THE INVENTION

The present inventors have now (1) identified a neoepitope that could serve as a biomarker for CC16 degradation by human neutrophil elastase; (2) developed and validated a competitive enzyme-linked immunosorbent assay (ELISA) specific for the identified neoepitope; and (3) evaluated and demonstrated the biological relevance of said assay in detecting the presence and severity of chronic obstructive pulmonary disease (COPD) and idiopathic pulmonary fibrosis (IPF).

Accordingly, in a first aspect the present invention provides a method of immunoassay, said method comprising contacting a patient sample with a monoclonal antibody that specifically binds to an HNE-generated neo-epitope of an HNE-generated fragment of club cell secretory protein 16 (CC16), and detecting and determining the amount of binding between said monoclonal antibody and peptides in the sample, wherein the HNE-generated neo-epitope consists of an N-terminus or C-terminus sequence of the HNE-generated fragment at an end of the HNE-generated fragment that has been cleaved by HNE.

In a preferred embodiment, the method of immunoassay comprises;
i) contacting a patient sample with a monoclonal antibody that specifically binds to the N-terminus amino acid sequence VDTLPQKPRE (SEQ ID NO: 1) or that specifically binds to the N-terminus amino acid sequence AMELFSPDQD (SEQ ID NO: 2) (said sequences also being referred to herein as “target sequences” or “CC16-HNE target sequences”); and
ii) detecting and determining the amount of binding between said monoclonal antibody and peptides in the sample.

Most preferably, step (i) comprises contacting the patient sample with a monoclonal antibody that specifically binds to the N-terminus amino acid sequence VDTLPQKPRE (SEQ ID NO: 1).

In a preferred embodiment, the method of immunoassay is a method of immunoassay for detecting and/or monitoring chronic obstructive pulmonary disease (COPD) or idiopathic pulmonary fibrosis (IPF) or a particular level of severity thereof in a patient, the method further comprising correlating said amount of binding with values associated with normal healthy subjects and/or values associated with known disease severity and/or values obtained from said patient at a previous time point and/or with a predetermined cut-off value. Most preferably, the method is a method for detecting and/or monitoring COPD or a particular level of severity thereof in a patient; a method for detecting and/or monitoring a particular level of severity of IPF in a patient; or a method for detecting and/or monitoring IPF with pulmonary hypertension (PH) in a patient.

Where the method is a method for detecting a particular level of severity of disease, the method may for example be a method for detecting very severe (GOLD stage 4) COPD or detecting IPF patients at an elevated risk of mortality.

In a preferred embodiment, the patient sample is a human biofluid sample. Preferably the sample is a blood-based sample, such as blood (whole blood), plasma or serum.

Where the monoclonal antibody is a monoclonal antibody that specifically binds to the N-terminus amino acid sequence VDTLPQKPRE (SEQ ID NO: 1), the monoclonal antibody preferably does not specifically bind to a peptide having the N-terminus amino acid sequence LVDTLPQKPRE (SEQ ID NO: 3) (i.e. an elongated version of the target sequence extended at its N-terminus by the addition of a leucine residue) and/or does not specifically bind to a peptide having the N-terminus amino acid sequence DTLPQKPRE (SEQ ID NO: 4) (i.e. a truncated version of the target sequence truncated by removal of the first valine residue). Preferably, the ratio of the affinity of said antibody for said target sequence to the affinity of said antibody for the elongated version of the target sequence is at least 10 to 1, and more preferably is at least 20 to 1, at least 30 to 1, at least 40 to 1, at least 50 to 1 or at least 100 to 1. Preferably, the ratio of the affinity of said antibody for said target sequence to the affinity of said antibody for the truncated version of the target sequence is at least 10 to 1, and more preferably is at least 20 to 1, at least 30 to 1, at least 40 to 1, at least 50 to 1 or at least 100 to 1.

Where the monoclonal antibody is a monoclonal antibody that specifically binds to the N-terminus amino acid sequence AMELFSPDQD(SEQ ID NO: 2), the monoclonal antibody preferably does not specifically bind to a peptide having the N-terminus amino acid sequence AAMELFSPDQD (SEQ ID NO: 5) (i.e. an elongated version of the target sequence extended at its N-terminus by the addition of an additional alanine residue) and/or does not specifically bind to a peptide having the N-terminus amino acid sequence MELFSPDQD (SEQ ID NO: 6) (i.e. a truncated version of the target sequence truncated by removal of the first alanine acid residue). Preferably, the ratio of the affinity of said antibody for said target sequence to the affinity of said antibody for the elongated version of the target sequence is at least 10 to 1, and more preferably is at least 20 to 1, at least 30 to 1, at least 40 to 1, at least 50 to 1 or at least 100 to 1. Preferably, the ratio of the affinity of said antibody for said target sequence to the affinity of said antibody for the truncated version of the target sequence is at least 10 to 1, and more preferably is at least 20 to 1, at least 30 to 1, at least 40 to 1, at least 50 to 1 or at least 100 to 1.

Where the monoclonal antibody is a monoclonal antibody that specifically binds to the N-terminus amino acid sequence VDTLPQKPRE (SEQ ID NO: 1), the monoclonal antibody may for example be raised against a synthetic peptide having the N-terminus amino acid sequence VDTLPQKPRE (SEQ ID NO: 1); and where the monoclonal antibody is a monoclonal antibody specifically binds to the N-terminus amino acid sequence AMELFSPDQD (SEQ ID NO: 2), the monoclonal antibody may for example be raised against a synthetic peptide having the N-terminus amino acid sequence AMELFSPDQD (SEQ ID NO: 2). For example, the monoclonal antibodies may be raised by: (a) immunizing a rodent (or other suitable mammal) with a synthetic peptide comprising the N-terminus sequence VDTLPQKPRE (SEQ ID NO: 1) or AMELFSPDQD (SEQ ID NO: 2), which peptide may optionally be linked at its C-terminus to an immunogenic carrier protein (such as keyhole limpet hemocyanin (“KLH”)); (b) isolating and cloning a single antibody producing cell; and (c) assaying the resulting monoclonal antibodies to ensure that they have the desired specificity. An exemplary protocol of the development, production and characterization of suitable monoclonal antibodies is described in the Examples section, infra.

In certain exemplary embodiments, where the monoclonal antibody is a monoclonal antibody that specifically binds to the N-terminus amino acid sequence VDTLPQKPRE, the monoclonal antibody may preferably comprise one or more complementarity-determining regions (CDRs) selected from:

CDR-L1:
(SEQ ID NO: 7)
KSSQSLFNSGTQKNYLA
CDR-L2:
(SEQ ID NO: 8)
WASTRES
CDR-L3:
(SEQ ID NO: 9)
KQSYNLLT
CDR-H1:
(SEQ ID NO: 10)
DYSMH
CDR-H2:
(SEQ ID NO: 11)
WINTETGEPTYADDFKG
CDR-H3:
(SEQ ID NO: 12)
MITVAMDY

Preferably the monoclonal antibody comprises at least 2,3,4,5 or 6 of the above listed CDR sequences.

Preferably the monoclonal antibody has a light chain variable region comprising the CDR sequences:

CDR-L1:
(SEQ ID NO: 7)
KSSQSLFNSGTQKNYLA
CDR-L2:
(SEQ ID NO: 8)
WASTRES
and
CDR-L3:
(SEQ ID NO: 9)
KQSYNLLT.

Preferably the monoclonal antibody has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 13)
KSSQSLFNSGTQKNYLAWYQQKPGQSPKLLIYWASTRESGVPDRFTGSG
SGTDFALTITSVQAEDLAVYYCKQSYNLLT

Preferably the monoclonal antibody has a heavy chain variable region comprising the CDR sequences:

CDR-H1:
(SEQ ID NO: 10)
DYSMH
CDR-H2:
(SEQ ID NO: 11)
WINTETGEPTYADDFKG
and
CDR-H3:
(SEQ ID NO: 12)
MITVAMDY.

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 14)
DYSMHWVKQAPGKGLKWVGWINTETGEPTYADDFKGRFAFSLETSASTA
YLRVNNLKNEDTATYFCGTMITVAMDY

Preferably, the monoclonal antibody comprises the light chain variable region sequence:

(SEQ ID NO: 15)
DIVLSQSPSSLAVSAGEKVTMSCKSSQSLFNSGTQKNYLAWYQQKPGQS
PKLLIYWASTRESGVPDRFTGSGSGTDFALTITSVQAEDLAVYYCKQSY
NLLTFGAGTKLEL
(CDRs bold and underlined; Framework sequences in
italics)

and/or the heavy chain variable region sequence:

(SEQ ID NO: 16)
QIQLVQSGPELKKPGETVKISCKASGYTFTDYSMHWVKQAPGKGLKWVG
WINTETGEPTYADDFKGRFAFSLETSASTAYLRVNNLKNEDTATYFCGT
MITVAMDYWGQGTSVTVSS
(CDRs bold and underlined; Framework sequences in
italics)

As used herein, the framework amino acid sequences between the CDRs of an antibody are “substantially identical” or “substantially similar” to the framework amino acid sequences between the CDRs of another antibody if they have at least 70%, 80%, 90% or at least 95% similarity or identity. The similar or identical amino acids may be contiguous or non-contiguous. The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

As used herein the term “N-terminus” refers to an N-terminal peptide sequence at the extremity of a polypeptide, i.e. at the N-terminal end of the polypeptide, and is not to be construed as meaning in the general direction thereof. As used herein the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4 in the case of humans and IgG1, IgG2a, IgG2b, IgG2c and IgG3 in the case of mice. The monoclonal antibody may preferably be of the IgG isotype, including any one of the IgG subclasses.

The CDR of an antibody can be determined using methods known in the art such as that described by Kabat et al. Antibodies can be generated from B cell clones. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. Any suitable analytical method can be used for measuring the amount of binding. For example, an ELISA method can be used in which spectrophotometric analysis is used to measure the amount of binding both in the patient samples and when producing the calibration curve.

As used herein the term “predetermined cut-off value” means an amount of binding that is determined statistically to be indicative of a high likelihood of a disease (e.g. COPD or IPF) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or particular severity thereof.

As used herein, the term “values associated with normal healthy subjects” means standardised quantities of binding determined by the method described supra for samples from subjects considered to be healthy, i.e. without disease (i.e. without COPD or IPF); and the term “values associated with known disease severity” means standardised quantities of binding determined by the method described supra for samples from patients known to have disease (i.e. COPD or IPF) of a known severity.

In a second aspect, the present invention provides a method of treating chronic obstructive pulmonary disease (COPD) or idiopathic pulmonary fibrosis (IPF) in a patient in need thereof, the method comprising:
(a) carrying out a method of immunoassay for detecting COPD or IPF or a particular level of severity thereof in accordance with the first aspect of the present invention on a sample from a patient; and
(b) administering to the patient a therapy for the treatment of COPD or IPF if it is determined in step (a) that the patient has COPD or IPF or said particular level of severity thereof.

The therapy may be any therapy suitable for treating the disease (COPD or IPF) in question. The therapy may for example comprise or consist of one or more medicaments, one or more lifestyle changes, one or more surgeries or combinations thereof. Medicaments may be formulated for topical administration (e.g. as a cream, foam, gel, lotion, ointment or patch), enteral administration (e.g. oral, rectal or sublingual) or parental administration (e.g. via injection or inhalation). Surgeries may be curative surgeries, preventative surgeries, palliative surgeries and/or restorative surgeries.

For example, suitable therapies for COPD may comprise one or more of: ceasing smoking; pulmonary rehabilitation (exercise training, nutritional modulation, occupational therapy, education and psychosocial counselling); influenza and pneumococcal vaccinations; oxygen therapy (supplemental oxygen); bronchodilators, such as beta2-adrenergic agonists and/or anticholinergics, in either long-acting or short-acting forms, such as for example salbutamol, terbutaline, salmeterol, formoterol, indacaterol, ipratropium, tiotropium, aclidinium, umeclidinium bromide, glycopyrronium; corticosteroids (inhaled and/or oral); phosphodiesterase-4 inhibitors (PDE4 inhibitors), such as for example roflumilast; antibiotics, such as for example amoxicillin, doxycycline, azithromycin, fluoroquinolones, erythromycin; methylxanthines, such as for example theophylline; mucolytics, such as for example erdosteine, carbocisteine; and in rare cases surgeries such as a lung volume reduction surgery, bronchoscopic lung volume reduction, bullectomy, or lung transplantation.

Suitable therapies for IPF may comprise one or more of: oxygen therapy (supplemental oxygen); pulmonary rehabilitation (exercise training, nutritional modulation, occupational therapy, education and psychosocial counselling); influenza and pneumococcal vaccinations; pirfenidone; nintedanib; lung transplantation; and palliative use of opioids.

In a third aspect, the present invention provides a monoclonal antibody that specifically binds to the N-terminus amino acid sequence VDTLPQKPRE (SEQ ID NO: 1) or that specifically binds to the N-terminus amino acid sequence AMELFSPDQD (SEQ ID NO: 2).

The antibody according to the third aspect of the invention is, in particular, suitable for use in carrying out the methods of immunoassay according to the first aspect of the invention. Preferred embodiments and features of the antibody according to the third aspect will therefore be apparent from the above discussion of the preferred embodiments of the methods according to the first aspect.

In a fourth aspect, the present invention provides an immunoassay kit comprising a monoclonal antibody in accordance with the third aspect of the present invention, and at least one of:
a streptavidin coated well plate
a biotinylated peptide VDTLPQKPRE-L-Biotin (SEQ ID NO: 17) wherein L is an optional linker, or a biotinylated peptide AMELFSPDQD-L-Biotin (SEQ ID NO: 18) wherein L is an optional linker
a secondary antibody for use in a sandwich immunoassay
a calibrator protein comprising the N-terminus amino acid sequence VDTLPQKPRE (SEQ ID NO: 1) or a calibrator protein comprising the N-terminus amino acid sequence AMELFSPDQD(SEQ ID NO: 2)
an antibody biotinylation kit
an antibody HRP labelling kit
an antibody radiolabelling kit

The immunoassay kit according to the fourth aspect of the invention is, in particular, suitable for use in carrying out the method of immunoassay according to the first aspect of the invention. Further preferred embodiments and features of the immunoassay kit according to the fourth aspect will therefore be apparent from the above discussion of the preferred embodiments of the methods according to the first aspect.

### FIGURES

FIG. 1: A partial sequence alignment for human, rat and mouse club cell secretory protein 16 (CC16). The arrow indicates one of the cleavage sites in CC16 for human neutrophil elastase (HNE). The section highlighted in grey indicates the section of the human sequence identified by mass spectrometry analysis which was selected for development of the monoclonal antibody employed in the CC16-HNE assay (and the corresponding sections of the aligned rat and mouse sequences).

FIG. 2: Specificity of the CC16-HNE assay. The CC16-HNE assay was found to be neoepitope specific as it reacted with the selection peptide but not any of the deselection peptides.

FIG. 3: Clinical evaluation of the CC16-HNE assay. Serum levels of CC16-HNE in healthy (n=20), COPD (n=58) and IPF (n=99) subjects at baseline are shown. Data are presented as individual data points with indication of median and interquartile range. The data was analyzed by Kruskal-Wallis with Dunn's multiple comparisons test; **p<0.01; ****p<0.0001.

FIG. 4: Characterization of CC16-HNE in COPD. CC16-HNE serum levels were investigated for associations with clinical parameters for COPD patients. A) CC16-HNE serum levels in female vs. male sex. B) Scatterplot of CC16-HNE levels and age. C) CC16-HNE serum levels presented by GOLD stages (2=moderate, 3=severe, 4=very severe). D) Scatterplot of CC16-HNE levels and FVC % predicted. E) Scatterplot of CC16-HNE and FEV % predicted. Group comparisons were performed with Mann-Whitney test (A) or Kruskal-Wallis with Dunn's multiple comparisons test (C) and data are shown as Tukey's box plot, indicating interquartile range. Correlations were assessed by Spearman's rank correlation coefficient. FVC=forced vital capacity; FEV1=forced expiratory volume in one second; GOLD=global initiative for chronic obstructive lung disease (1=mild, 2=moderate, 3=severe, 4=very severe); ns=non-significant; SD=standard deviation; *p<0.05.

FIG. 5: Survival prediction and characterization of CC16-HNE in IPF. A) Serum CC16-HNE levels were assessed at baseline in IPF subjects (n=99) and dichotomized at the median. Patients with low CC16-HNE levels had significantly higher risk of all-cause mortality as compared to IPF patients with high CC16-HNE levels with a hazard ratio of 1.79 (95% CI 1.03-3.13; p=0.04). B) CC16-HNE serum levels in female vs. male sex. C) Scatterplot of CC16-HNE levels in IPF patients and age. D) Scatterplot of CC16-HNE levels in IPF patients and FVC (% predicted). Group comparisons were performed with Mann-Whitney test and data are shown as Tukey's box plot, indicating interquartile range. Correlations were assessed by Spearman's rank correlation coefficient. FVC=forced vital capacity; ns=non-significant.

FIG. 6: Direct comparison of the CC16-HNE assay and an CC16 assay. A) CC16-HNE serum levels in healthy (n=20), COPD (n=20), and IPF (n=19) subjects. B) CC16-HNE ROC curve for separation of healthy and COPD subjects. C) CC16-HNE ROC curve for separation of healthy and IPF subjects. D) CC16 serum levels in healthy, COPD, and IPF subjects. E) CC16 ROC curve for separation of healthy and COPD subjects. F) CC16 ROC curve for separation of healthy and IPF subjects. Data are shown as individual data points with indication of median and interquartile range and analyzed by Kruskal-Wallis with Dunn's multiple comparisons test. AUC=area under the curve; ns=non-significant; ROC=receiver operating characteristics; **p<0.01; ****p<0.0001.

FIG. 7: CC16-HNE levels were measured in serum samples collected from idiopathic pulmonary fibrosis (IPF) subjects with (IPF+PH, n=13) or without (IPF-PH, n=86) pulmonary hypertension (PH). Biomarker levels were log-transformed and analyzed by a linear regression model adjusted for age and sex. Data are shown as the estimated geometric mean±95% confidence intervals (CI) back-transformed. ** indicates significant difference between PH negative and PH positive IPF subjects (p-value<0.01).

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

All reagents used were high quality chemicals from Merck (Whitehouse Station, NJ, USA) and Sigma (St. Louis MO, USA) unless stated otherwise. All synthetic peptides (Table 1) were purchased from Genscript (Piscataway, NJ, US).

### In Vitro Cleavage and Target Identification

Recombinant CC16 (uteroglobin SCGB1A1) (cat. no. MBS691837, MyBioSource, San Diego, CA, US) was cleaved in vitro with native human neutrophil elastase (cat. no. ab91099, Abcam, Cambridge, UK) in a ratio of 10:1 in cleavage buffer (50 mM Tris-HCl, 150 mM NaCl, pH 7.5) in low binding Eppendorf tubes for 3 h/24 h at 37° C. Cleavage was confirmed by silver staining using the SilverXpress kit (Invitrogen) according to manufacturer's instructions, and samples were stored at −80° C. Thereafter, cleavage samples were prepared for mass spectrometry analysis. In brief, samples were passed through a 30K molecular weight cut-off (MWCO) filter (30 kD filter from Pall, 30K, P/N OD030C34). Then, samples were desalted by Vydac UltraMicro Spin C18 columns (cat. no. 74-7206, Harvard Apparatus). When bound on the column material, the samples were rinsed with 2% acetonitrile in 0.2% formic acid, whereafter these were eluded with 50% acetonitrile in 0.1% formic acid. Samples were transferred to vials, dried by Speedvac, and stored at −20° C. until reconstituted and analyzed.

Peptides were identified from raw data by Mascot 2.2 in the Proteome Discoverer software 1.4 (Thermo Fisher Scientific). Peptides with a probability score of p<0.05 were chosen for further analysis. CC16 peptides identified in the CC16/HNE sample, and not overlapping with peptides in a control sample without HNE, were considered as HNE derived CC16 degradation fragments. The ten amino acids at the C- or N-terminus of each peptide at the end(s) cleaved by HNE were considered to contain the HNE-generated neoepitopes, with each neoepitope being expected to comprise the first six amino acids at the C- or N-terminus of the peptide at the end cleaved by HNE (since an antibody specific for one of said HNE-generated neoepitopes would be expected to rely on binding to said first six amino acids). Thus, each of said six amino acid sequences was analyzed for uniqueness by protein blasting using the NPS@: Network Protein Sequence Analysis tool [14] followed by analysis for species homology using the UniProt sequence alignment tool [15].

### Monoclonal Antibody Development, Production, and Characterization

A monoclonal antibody (mAb) specific for the N-terminus amino acid sequence ⁶⁵↓⁶⁶VDTLPQKPRE (SEQ ID NO: 1) (identified and selected following HNE cleavage of CC16 and analysis of the fragments via the procedure described above) was raised using the following procedure. Five female Balb/C mice of 6-7 weeks of age were immunized subcutaneously with 200 μl emulsified antigen and 100 μg immunogenic peptide (VDTLPQKPRE-GGC-“KLH” (SEQ ID NO: 19)) using Stimmune (Thermo Fisher). The immunizations were repeated every second week until stable serum antibody titer levels were reached. The mouse with the highest serum titer was chosen for fusion and rested for a month. Then, the selected mouse was boosted intravenously with 50 μg immunogenic peptide in 100 μl 0.9% NaCl solution 3 days before isolation of the spleen for cell fusion. The mouse spleen cells were fused with SP2/0 myeloma cells to produce hybridoma cells as described by Gefter et al. [16]. Hybridoma cells were plated in individual wells into 96-well microtiter plates for further growth using the limiting dilution method to promote monoclonal growth. An indirect ELISA performed on streptavidin-coated 96-well microtiter plates was used to screen supernatants from hybridoma clones for reactivity to identify the best mAb-producing clones. The assay employed a biotin-labeled screening peptide (VDTLPQKPRE-K-Biotin (SEQ ID NO: 10)) and tested antibody specificity using selection (VDTLPQKPRE (SEQ ID NO: 1)) and deselection (elongated [LVDTLPQKPRE (SEQ ID NO: 3)], truncated [DTLPQKPRE (SEQ ID NO: 4)], and nonsense [QMAGLDEKSG (SEQ ID NO: 21)]) peptides (see Table 1). The best clone was selected based on specificity to the selection peptide and no reaction to the deselection peptides. Supernatant was harvested and mAb was purified using HiTrap affinity columns (GE Healthcare Life Science, Little Chalfront, Buckinghamshire, UK) whereafter the mAb was labeled with horseradish peroxidase (HRP) using the using the Roche Peroxidase Labeling Kit (cat. no. 11829696001, Merck). Finally, antibody isotype was determined using the Rapid ELISA Mouse mAb Isotyping Kit (Invitrogen, Carlsbad, CA, USA). All procedures were performed according to the manufacturers' instructions.

TABLE 1
Synthetic peptides used for antibody production
and assay development.

| Synthetic peptide | Amino acid sequence |
|---|---|
| Immunogenic peptide | VDTLPQKPRE-GGC-KLH |
| | (SEQ ID NO: 19) |
| Screening peptide | VDTLPQKPRE-K-biotin |
| | (SEQ ID NO: 20) |
| Selection peptide | VDTLPQKPRE |
| | (SEQ ID NO: 1) |
| Deselection peptides | |
| Elongated | LVDTLPQKPRE |
| | (SEQ ID NO: 3) |
| Truncated | DTLPQKPRE |
| | (SEQ ID NO: 4) |
| Nonsense 1 | QMAGLDEKSG |
| | (SEQ ID NO: 21) |
| Nonsense 2 | QAAFSQYKKV |
| | (SEQ ID NO: 22) |
| Nonsense screening | biotin-QAAFSQYKKV |
| peptide | (SEQ ID NO: 23) |

### CC16-HNE Assay Development

The development of a competitive ELISA employing the HRP-labeled mAb described above (referred to hereinafter as the “CC16-HNE assay”) included several preliminary optimizing experiments where reagents, concentrations, incubation-time and -temperature were analyzed in addition to testing reactivity to selection and deselection peptides (Table 1) and human sera. The final CC16-HNE assay procedure was as follows: A 96-well streptavidin-coated microtiter plate (cat. no. 11940279, Roche Diagnostics, Hvidovre, Denmark) was coated with 100 μL/well of 15 ng/mL screening peptide (VDTLPQKPRE-K-biotin (SEQ ID NO: 20)) diluted in coating buffer (25 mM phosphate buffered saline (PBS) with 1% bovine serum albumin, 0.1% Tween-20, 0.36% Bronidox (BTB), 4 g/L NaCl, pH 7.4) and incubated for 30 minutes at 20° C. Next, 20 μL/well of selection peptide (two-fold dilution from 1000 ng/mL), assay controls, and samples of interest were added to the appropriate wells in double determinations, followed by the addition of 100 μL/well of 180 ng/mL HRP-labeled mAb diluted in assay buffer (25 mM PBS-BTB, 4 g/L NaCl, 5% Liquid II, pH 7.4) and plates were incubated for 20 hours at 4° C. The colorimetric substrate 3,3′,5,5′-tetramethylbenzidine (TMB) One (cat. no. 4380H, Kem-En-Tec, Taastrup, Denmark) was added 100 μL/well and incubated for 15 min whereafter the reaction was stopped using 100 μL/well of 0.1% sulphuric acid. The absorbance was measured at wavelength 450 nm with 650 as reference on a microplate reader (VersaMax, Molecular Devices, Sunnyvale, CA, USA). All incubation steps were performed in the dark with constant shaking at 300 rpm and followed by five times wash of the wells using washing buffer (20 nM Tris, 50 mM NaCl, pH 7.2). A 10-point calibration curve was plotted using a 4-parameter logistic curve fit of the selection peptide dilutions. Data were analyzed using the SoftMax Pro version 7.1.0 software (Molecular Devices).

### Technical Evaluation

Antibody specificity was evaluated using synthetic peptides and calculated as percentage of signal inhibition of two-fold diluted selection and deselection peptides (Table 1). Unspecific binding was tested using a nonsense screening peptide (biotin-QAAFSQYKKV (SEQ ID NO: 23)). IC50 (half-maximal inhibition concentration) was determined from 10 independent runs of the 10-point calibration curve. The technical lower limit of detection (LLOD) was calculated as the mean signal of 60 determinations of a blank sample (i.e., assay buffer) plus 3× standard deviation (SD). The upper limit of quantification (ULOQ) was determined from 10 independent runs of the calibration curve and determined as the highest concentration of selection peptide determined with a recovery percentage within 100±20% of the nominal concentration and a CV<20%. The lower limit of quantification (LLOQ) was determined for serum by triplicate assessment of five independent runs using four human serum samples (total of 15 determinations per sample), covering the lower range of the calibration curve. Mean concentration and CV % for each sample were plotted and a regression model was used to determine the LLOQ as the lowest analyte concentration where the CV % with 95% confidence equals 20%. Two-fold dilutions of four human serum samples quantified in three independent runs were used to evaluate assay linearity, calculated as percentage of recovery of the undiluted sample. The intra- and inter-assay variation was determined by 10 independent runs of 10 quality control samples (eight human serum and two assay controls) run in double determinations. Assay stability was evaluated by storing assay reagents at 20° C. for 24 hours and at 37° C. for 24, 72 or 168 hours. The 10 quality control samples were quantified in three independent runs using stressed assay reagents and recovery percentage was calculated using mean concentrations obtained in the intra- and inter-variation test as references.

Accuracy of the assay was assessed in three human serum samples spiked with two-fold dilutions of the selection peptide and three human serum samples spiked with two-fold dilutions of human serum samples with high concentration of the analyte. Percentage recovery was calculated for the spiked samples using the theoretical amount of analyte in the sample as reference. Analytical interference was evaluated by adding a low/high concentration of haemoglobin (2.5/5.0 mg/mL), lipids (1.5/5.0 mg/mL) or biotin (5.0/40 ng/mL) to three human serum samples of known concentration and calculating the recovery percentage in the spiked sample with the un-spiked sample as reference. Analyte stability was determined in human serum samples stressed by up to five freeze/thaw cycles or by storage at 4° C. or 20° C. for 2, 4, 24, or 48 hours. Recovery was calculated with unstressed samples as reference. All tests used double determinations of samples unless otherwise stated.

### Clinical Evaluation of the CC16-HNE Assay

The clinical relevance of the novel CC16-HNE assay (described above) was evaluated in serum samples from COPD, IPF, and healthy subjects. Measurements of serum samples were performed in double determinations and values outside the measurements range were assigned the value of LLOQ or ULOQ as appropriate. Healthy subjects were obtained from a commercial vendor (Discovery Life Science, Los Osos, CA). COPD subjects originated from an observational study described previously [17]. Inclusion criteria for COPD subjects were a COPD diagnosis by a senior physician and FEV1<80% of predicted and exclusion were acute exacerbation resulting in hospitalization four weeks prior to blood sampling. IPF subjects had prevalent disease and were part of a larger study described previously [18]. IPF subjects were >18 years of age, diagnosed with IPF, and excluded if linguistic or intellectual barriers prevented completion of questionnaires. Clinically relevant parameters were collected at baseline for both disease groups. Additionally, mortality data was recorded over a five-year period for the IPF subjects.

For clinical evaluation, CC16-HNE levels (i.e. the levels of peptide detected and measured by the CC16-HNE assay) were measured in serum from healthy (n=20), COPD (n=58) and IPF (n=99) subjects collected at study baselines. For a head-to-head comparison with a commercial non-neoepitope assay for CC16, CC16 levels (i.e. the levels of CC16 as detected and measured by said commercial assay) and CC16-HNE levels were directly compared in healthy (n=20), COPD (n=20), and IPF (n=19) subjects collected at a follow-up visit. Said commercial human CC16 ELISA (cat. no. RD191022200, BioVendor, Brno, Czech Republic) was run according to manufacturer's instructions with samples in double determinations.

In a further clinical evaluation, CC16-HNE levels were also measured in serum samples from IPF subjects diagnosed with (n=13) and without (n=86) PH.

### Ethical Statement

All animals were treated according to the guidelines for animal welfare. Antibody production in mice was approved by the Danish National Authority (The Animal Experiments Inspectorate) under approval number 2013-15-2934-00956. The collection and retrieval of the human serum complied with international ethical guidelines for handling human sample and patient information. All participants signed an informed consent, and the study was approved by the local ethical committee. Samples were collected after informed consent and approval by the local Ethical Committee and in compliance with the Helsinki Declaration of 1975.

### Statistical Analysis

CC16-HNE and CC16 serum levels were analyzed by Kruskal-Wallis test with Dunn's multiple comparisons test. Separation of disease groups from healthy (diagnostic accuracy) was further evaluated by assessing the area under the curve (AUC) by receiver operations characteristics (ROC). Correlations were assessed by Spearman's rank correlation coefficient and statistical significance between sexes were assessed by Mann-Whitney U test. Interquartile range (IQR) of biomarker levels and 95% confidence interval (95% CI) of estimations are presented as appropriate. P-values below 0.05 were considered statistically significant. Statistical analysis and graphs were performed using GraphPad Prism version 9.5.0 (GraphPad Software, Inc., La Jolla, CA).

### Results

### CC16-HNE Neoepitope Identification and mAb Development

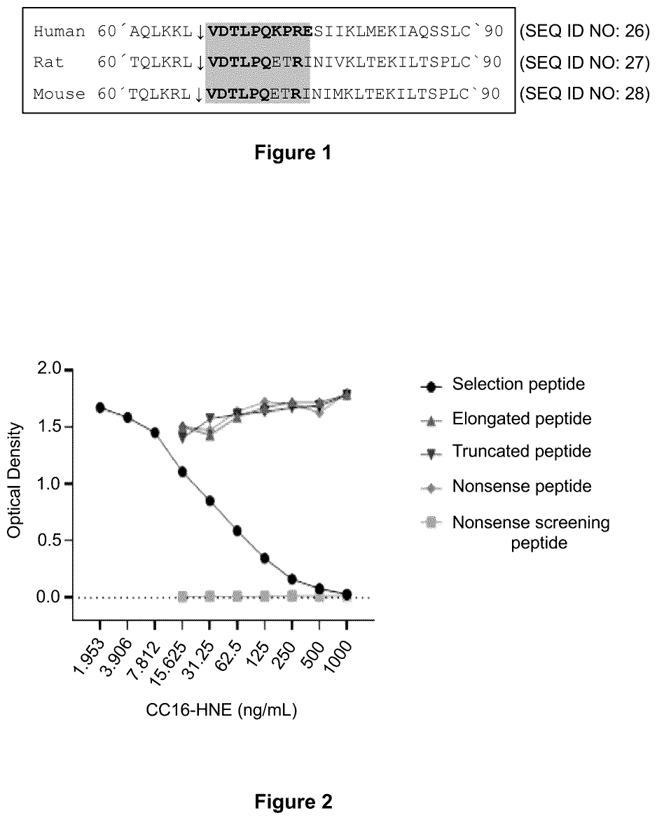

From the peptides identified by MS analysis, two peptides with unique amino acid sequences released from HNE-mediated cleavage of CC16 in vitro were selected as potential candidates: ⁴⁴↓⁴⁵AMELFSPDQD (SEQ ID NO: 2) and ⁶⁵↓⁶⁶VDTLPQKPRE (SEQ ID NO: 1). Due to a higher degree of species homology between human, rat, and mouse, the N-terminus amino acid sequence ⁶⁶↓⁶⁶VDTLPQKPRE (SEQ ID NO: 1) was selected for antibody development. The human, rat, and mouse sequences were 100% identical at the first six amino acids closest to the cleavage site, with the rat and mouse sequences differing from the human sequence at amino acid positions 7, 8 and 10 (FIG. 1).

Mice were immunized with the immunogenic peptide (VDTLPQKPRE-GGC-“KLH” (SEQ ID NO: 19)) comprising said chosen N-terminus amino acid sequence, and the resulting mAb with the highest selectivity for the selection peptide (VDTLPQKPRE (SEQ ID NO: 1)) and the best native reactivity and stability was chosen for assay development, as described supra.

The isotype, sequence and CDRs of this monoclonal antibody were determined. The sequence of the chains are as follows (CDRs underlined and in bold; N-terminus signal peptide and C-terminus Constant region in italics):

Heavy Chain Sequence (Mouse IgG2b isotype)
(SEQ ID NO: 24)
MAWVWTLLFLMAAAQSIQAQIQLVQSGPELKKPGETVKISCKASGYTFT
DYSMHWVKQAPGKGLKWVGWINTETGEPTYADDFKGRFAFSLETSASTA
YLRVNNLKNEDTATYFCGTMITVAMDYWGQGTSVTVSSAKTTPPSVYPL
APGCGDTTGSSVTLGCLVKGYFPESVTVTWNSGSLSSSVHTFPALLQSG
LYTMSSSVTVPSSTWPSQTVTCSVAHPASSTTVDKKLEPSGPISTINPC
PPCKECHKCPAPNLEGGPSVFIFPPNIKDVLMISLTPKVTCVVVDVSED
DPDVQISWFVNNVEVHTAQTQTHREDYNSTIRVVSTLPIQHQDWMSGKE
FKCKVNNKDLPSPIERTISKIKGLVRAPQVYILPPPAEQLSRKDVSLTC
LVVGENPGDISVEWTSNGHTEENYKDTAPVLDSDGSYFIYSKLNMKTSK
WEKTDSFSCNVRHEGLKNYYLKKTISRSPGK
Light Chain Sequence (Mouse Kappa Isotype)
(SEQ ID NO: 25)
MDSQAQVLILLLLWVSGTCGDIVLSQSPSSLAVSAGEKVTMSCKSSQSL
FNSGTQKNYLAWYQQKPGQSPKLLIYWASTRESGVPDRFTGSGSGTDFA
LTITSVQAEDLAVYYCKQSYNLLTFGAGTKLELRRADAAPTVSIFPPSS
EQLTSGGASVVCFLNNFYPKDINVKWKIDGSERQNGVLNSWTDQDSKDS
TYSMSSTLTLTKDEYERHNSYTCEATHKTSTSPIVKSFNRNEC

### Technical Evaluation and Characterization of the CC16-HNE Assay

An assay employing the selected mAb was developed on the competitive ELISA platform and named CC16-HNE, as described supra. To evaluate the specificity of the assay, the reactivity towards selection and deselection peptides was evaluated (see Table 1 for peptide details). The CC16-HNE assay was found to only detect the selection peptide and not the deselection peptides, indicating that the assay was specific to the HNE-generated CC16 neoepitope (FIG. 2).

A summary of the technical evaluation of the CC16-HNE assay can be found in Table 2. Briefly, the measurement range (LLOQ-ULOQ) was determined to 13.4-1000 ng/mL and the IC50 was 36.9 ng/ml. The intra- and inter assay variation was 7.1% and 11.6%, respectively, and linearity was accepted from undiluted to a 2- and 4-fold dilution of human serum (104.3-106.5% recovery). The assay remained stable after prolonged storage of kit reagents at up to 37° C. for 7 days (100.8-119.2% recovery). The analyte in human serum remained stable after stressing samples for up to five freeze/thaw cycles (98.5-113.5% recovery) and following prolonged storage of samples for up to 48 hours at 20° C. (87.2-107.5%). Haemoglobin and lipids did not interfere with measurements of CC16-HNE levels while a high, but not low, concentration of biotin showed mild interference with recovery of the analyte just outside the accepted limit of 120% (121.7% recovery). The CC16-HNE assay showed good accuracy as shown by recovery of human serum spiked with the selection peptide or another serum sample of 87.3-104.1%.

TABLE 2
Recovery percentage (REC %) is
accepted when within 100 ± 20%.

| | |
|---|---|
| Assay parameters | Results |

| Assay format | Competitive ELISA with | |
|---|---|---|
| | colorimetric detection; | |
| | Sample incubation: 20 hrs at 4° C. | |
| Measurement range, serum | 13.4-1000 | ng/mL |
| (LLOQ-ULOQ) | | |
| Technical LLOD | 8.2 | ng/mL |
| IC50 | 36.9 | ng/ml |

| | |
|---|---|
| Assay linearity (human | |
| serum, REC %) | |
| 1 + 1 | 104.3% |
| 1 + 3 | 106.5% |
| 1 + 7 | 132.7% |
| Intra-assay variation | 7.1% |
| (CV %) | |
| Inter-assay variation | 11.6% |
| Assay stability (REC %) | |
| 20° C. (24 h) | 100.8% |
| 37° C. (24/72/168 h) | 103.6/99.9/119.2% |
| Accuracy (REC %) | |
| Peptide in serum | 87.3% |
| Serum in serum | 104.1% |
| Analytical interference | |
| (REC %) | |
| Haemoglobin (low/high) | 99/116% |
| Lipids (low/high) | 99/103% |
| Biotin (low/high) | 109.6/121.7% |
| Analyte stability (REC %) | |
| 4° C. (2/4/24/48 h) | 90.3/92.9/87.2/88.1% |
| 20° C. (2/4/24/48 h) | 98.3/101.9/107.5/105.8% |
| Freeze/thaw (cycle | 113.5/98.5/98.7/102.1/113.5% |
| 1/2/3/4/5) | |

CV %, coefficient of variance percentage; LLOD, lower limit of detection; LLOQ, lower limit of quantification; ULOQ, upper limit of quantification.

### Clinical Evaluation of the CC16-HNE Assay

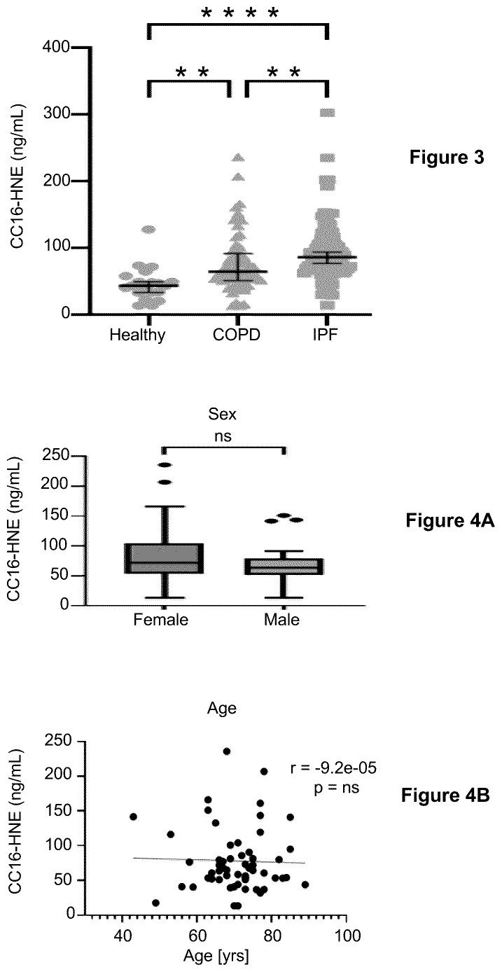

Demographics for the initial patient cohorts used to evaluate the clinical utility of CC16-HNE are summarized in Table 3. Serum CC16-HNE levels were determined in healthy, COPD, and IPF subjects and were found to be significantly elevated in both COPD (median 64.2 [IQR 51.1-92.2] ng/mL, p=0.0047) and IPF (median 85.5 [IQR 66.6-108.5] ng/mL, p<0.0001) when compared to healthy subjects (median 43.16 [IQR 33.0-55.5] ng/mL) (FIG. 3). Additionally, a statistically significant difference in CC16-HNE levels was found between COPD and IPF patients (p=0.0161).

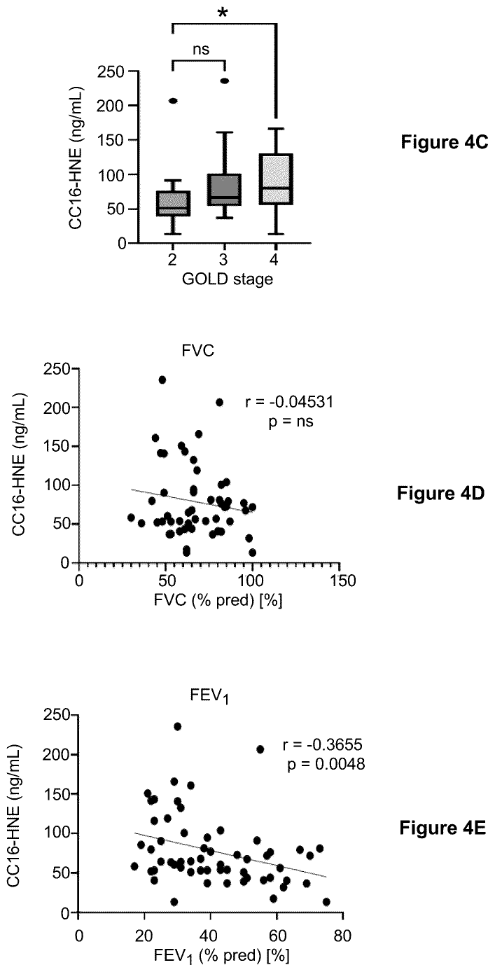

In the COPD cohort, CC16-HNE levels were not associated with age, sex, or forced vital capacity (FVC), but were significantly inversely correlated with FEV1 (r=−0.366, p=0.0048) (FIGS. 4A-B and 4D-E). A slight but statistically significant increase in CC16-HNE was seen for very severe COPD patients (GOLD stage 4, median 79.9 [IQR 56.0-130.5] ng/mL) when compared to patients with moderate COPD (GOLD stage 2, median 51.0 [IQR 39.4-76.4] ng/mL; p=0.049) (FIG. 4C).

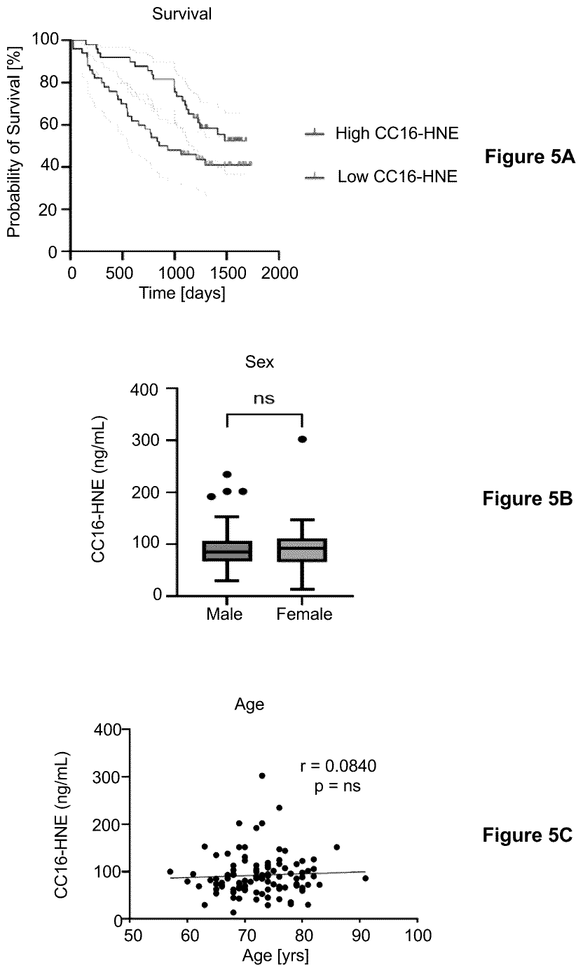

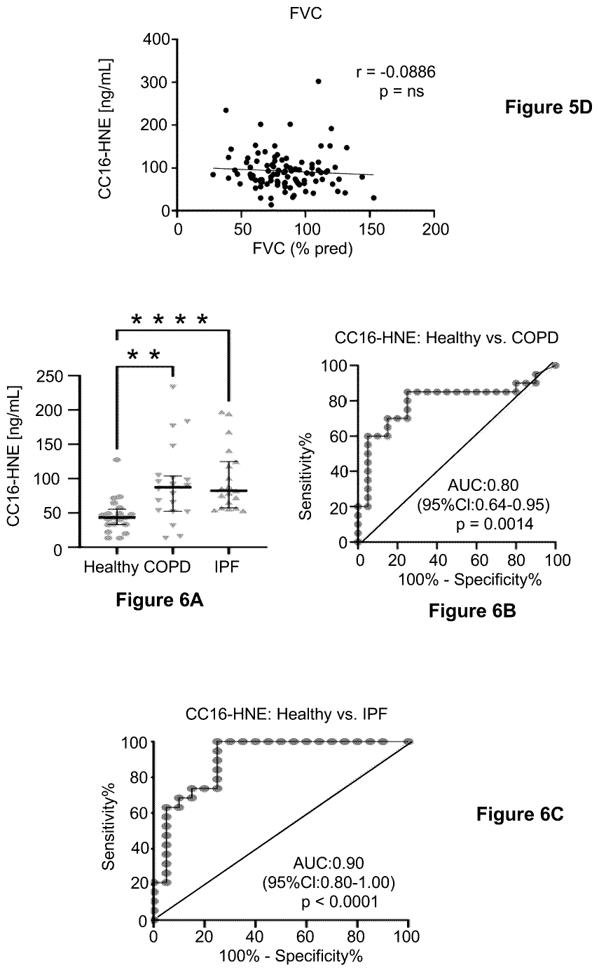

In the IPF cohort, serum levels of CC16-HNE were prognostic for mortality outcome. Patients with baseline CC16-HNE levels below the median had a significantly elevated risk of mortality within five years as compared to those with levels above the median (hazard ratio 1.79 [95% CI 1.03-3.13]; p=0.04) (FIG. 5A). In these patients, CC16-HNE was not associated with sex (males: median 84.96 [IQR 66.6-107.2]ng/mL; females: median 91.7 [IQR 65.4-112.0] ng/mL) (FIG. 5B), age (FIG. 5C), or FVC (FIG. 5C) at time of blood collection.

TABLE 3
Demographics of clinical cohorts.

| | | |
|---|---|---|
| Healthy | COPD | IPF |

Clinical evaluation of CC16-HNE

| n | 20 | | 58 | | 99 | |
|---|---|---|---|---|---|---|
| Age (years), mean | 56.6 | (7.2) | 70.7 | (8.7) | 72.6 | (6.0) |
| (SD) | | | | | | |
| Male sex, n (%) | 9 | (45.0%) | 24 | (41.4%) | 79 | (79.8%) |

| Smoking status | — | (3/46/9) | | (27/67/5) | |
|---|---|---|---|---|---|
| (never/former/current) | | | | | |
| GOLD stage (1/2/3/4) | — | (0/19/22/17) | | — | |
| FEV₁ (% predicted), | — | 40.8 | (15.7) | — | |
| mean (SD) | | | | | |
| FVC (% predicted), | — | 67.4 | (17.4) | 83.9 | (24.7) |
| mean (SD) | | | | | |
| Emphysema, n (%) | — | 3 | (5.2%) | 48 | (48.9%) |

Diagnostic potential: CC16-HNE vs. CC16

| n | 20 | | 20 | | 19 | |
|---|---|---|---|---|---|---|
| Age (years), mean | 56.6 | (7.2) | 71.5 | (7.9) | 75.5 | (5.2) |
| (SD) | | | | | | |
| Male sex, n (%) | 9 | (45.0%) | 11 | (55%) | 15 | (78.9%) |

| Smoking status | — | (1/18/1) | | (5/13/1) | |
|---|---|---|---|---|---|
| (never/former/current) | | | | | |
| GOLD stage (1/2/3/4) | | (0/3/7/10) | | — | |
| FEV₁ (% predicted) | — | 33.6 | (13.9) | — | |
| FVC (% predicted) | — | 60.9 | (14.0) | 90.1 | (26.3) |
| Emphysema, n (%) | — | 0 | (0.0%) | 13 | (68.4%) |

FVC, forced vital capacity; FEV₁, forced expiratory volume in one second; GOLD, global initiative for chronic obstructive lung disease (1 = mild, 2 = moderate, 3 = severe, 4 = very severe); SD, standard deviation.

In the cohorts of IPF patients with (n=13) and without (n=86) PH, it was found that baseline CC16-HNE serum levels were lower in patients with PH (PH positive, adjusted mean 58.4 [95% CI 45.4-75.2] ng/mL) compared to patients without PH (PH negative, adjusted mean 87.7 [95% CI 77.5-99.1] ng/mL; p=0.004) (FIG. 7).

### Diagnostic Potential of the CC16-HNE Assay Vs. a CC16 Assay

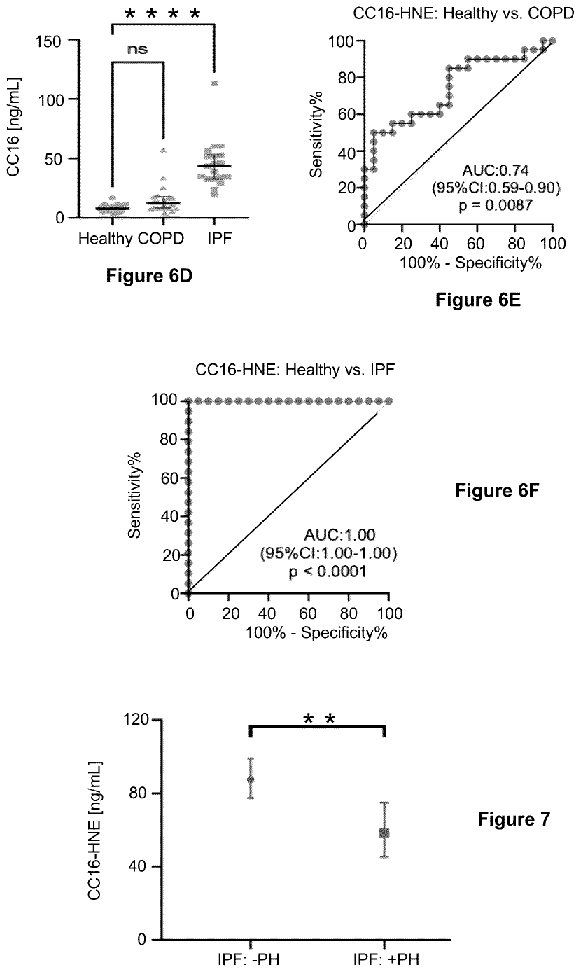

Performance of the CC16-HNE assay was compared directly to that of a commercial assay for intact CC16 in a head-to-head comparison measuring healthy, COPD, and IPF subjects. CC16-HNE serum levels were significantly elevated in both COPD (median 87.3 [IQR 52.3-104.9] ng/mL; p=0.0018) and IPF (median 82.46 [IQR 57.4-124.9] ng/mL; p=0.0001) compared to healthy subjects (median 43.2 [IQR 33.0-55.5] ng/mL) (FIG. 6A). For serum CC16 there was no statistical difference in levels between healthy (median 7.78 [IQR 6.14-9.48] ng/mL) and COPD (median 12.34 [IQR 8.18-17.83] ng/mL; p=0.092) but statistical significance was reached for IPF (median 43.51 [IQR 32.7-52.85]; p<0.0001) compared to healthy subjects (FIG. 6D). Accordingly, the separation between healthy and COPD subjects was bigger for CC16-HNE (AUC 0.80 [95% CI 0.64-0.95]; p=0.0014) (FIG. 6B) than for CC16 (AUC 0.74 [95% CI 0.59-0.90]; p=0.0087) (FIG. 6E). The separation between healthy and IPF subjects was similar and highly significant for CC16-HNE (AUC 0.90 [95% CI 0.80-1.00]; p<0.0001) (FIG. 6C) and CC16 (AUC 1.00 [95% CI 1.00-1.00]; p<0.0001) (FIG. 6F).

### Discussion

In this study, the inventors have developed and characterized a competitive ELISA, the “CC16-HNE assay”, that can quantify the levels (in serum or other biofluids) of a neoepitope of CC16 generated by cleavage of CC16 by HNE. The inventors have shown that the assay is specific to the targeted neoepitope, accurate, precise, and stable.

CC16 has previously been proposed as a biomarker for COPD and IPF, but here the inventors have presented data indicating that the CC16-HNE assay developed by the present inventors (which measures the levels of an HNE-generated neoepitope of CC16) is superior for separating COPD and healthy subjects than commercially available CC16 assays (that measure the levels of intact CC16). Furthermore, the CC16-HNE assay showed good separation of IPF and healthy subjects and baseline levels in IPF subjects were prognostic for mortality outcome. The CC16-HNE assay was also able to distinguish between IPF subjects with and without PH.

The CC16-HNE assay measures the proteolytic activity of HNE on CC16 to quantify a specific process related to lung inflammation. CC16 is believed to be released in response to inflammation or airway epithelial damage while HNE is released from incoming neutrophils as part of a response to inflammation. Thus, CC16-HNE levels reflect lung inflammation by assessing in combination two highly relevant processes in a unique way, providing additional information compared to the standard CC16 assay, and being easily quantifiable in serum by the novel CC16-HNE assay. In addition, it has been highlighted that CC16 could have potential as an early screening marker for lung diseases due to its essential role in modulation of respiratory disease and abundance [7]. In this study, the inventors showed that assessing the combination of two disease-relevant processes improves the diagnostic value for COPD as compared to assessing intact CC16 alone. Additionally, the levels of the CC16-HNE neoepitope were not affected by age or sex, indicating that serological levels reflect the inflammatory status of the subject's lungs.

In conclusion, the inventors have developed a novel method for quantifying lung inflammation in serum by measuring a unique fragment of CC16 generated by HNE. The CC16-HNE assay was robust and found to be a biomarker assay with prognostic and diagnostic capabilities in COPD and IPF subjects.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

[1]W. W. Labaki and M. K. Han, “Chronic respiratory diseases: a global view,” The Lancet Respiratory, vol. 8, pp. 531-533, 2020, doi: 10.1016/S2213-2600(19)30356-X.
[2]S. Safiri et al., “Burden of chronic obstructive pulmonary disease and its attributable risk factors in 204 countries and territories, 1990-2019: results from the Global Burden of Disease Study 2019,” BMJ, vol. 378, July 2022, doi: 10.1136/BMJ-2021-069679.
[3]T. M. Maher et al., “Global incidence and prevalence of idiopathic pulmonary fibrosis,” Respir Res, vol. 22, no. 1, p. 197, July 2021, doi: 10.1186/s12931-021-01791-z.
[4]B. Beghé, S. Cerri, L. M. Fabbri, and A. Marchioni, “COPD, Pulmonary Fibrosis and ILAs in Aging Smokers: The Paradox of Striking Different Responses to the Major Risk Factors,” Int J Mol Sci, vol. 22, no. 17, September 2021, doi: 10.3390/IJMS22179292.
[5]J. E. Boers, A. W. Ambergen, and F. B. J. M. Thunnissen, “Number and Proliferation of Clara Cells in Normal Human Airway Epithelium,” https://doi.org/10.1164/ajrccm.159.5.9806044, vol. 159, no. 5 I, pp. 1585-1591, December 2012, doi: 10.1164/AJRCCM.159.5.9806044.
[6]A. P. Wong, A. Keating, and T. K. Waddell, “Airway regeneration: The role of the Clara cell secretory protein and the cells that express it,” Cytotherapy, vol. 11, no. 6, pp. 676-687, 2009, doi: 10.3109/14653240903313974.
[7]S. Almuntashiri, Y. Zhu, Y. Han, X. Wang, P. R. Somanath, and D. Zhang, “Club Cell Secreted Protein CC16: Potential Applications in Prognosis and Therapy for Pulmonary Diseases,” J Clin Med, vol. 9, no. 12, pp. 1-16, December 2020, doi: 10.3390/JCM9124039.
[8]M. E. Laucho-Contreras et al., “Protective role for club cell secretory protein-16 (CC16) in the development of COPD,” Eur Respir J, vol. 45, pp. 1544-1556, 2015, doi: 10.1183/09031936.00010515.
[9]I. Buendía-Roldá et al., “Increased Expression of CC16 in Patients with Idiopathic Pulmonary Fibrosis,” 2016, doi: 10.1371/journal.pone.0168552.
[10]A. Bernard, F. X. Marchandise, S. Depelchin, R. Lauwerys, and Y. Sibille, “Clara cell protein in serum and bronchoalveolar lavage,” Eur Rasplr J, vol. 5, pp. 1231-1238, 1992.
[11]D. A. Lomas, E. K. Silverman, L. D. Edwards, B. E. Miller, H. O. Coxson, and R. Tal-Singer, “Evaluation of serum CC-16 as a biomarker for COPD in the ECLIPSE cohort,” 2008, doi: 10.1136/thx.2008.102574.
[12]S. Guerra et al., “Relation between circulating CC16 concentrations, lung function, and development of chronic obstructive pulmonary disease across the lifespan: a prospective study,” Elsevier, Accessed: Feb. 21, 2023. [Online]. Available: https://www.sciencedirect.com/science/article/pii/S2213260015 001964
[13] “Prednisone, Azathioprine, and N-Acetylcysteine for Pulmonary Fibrosis,” New England Journal of Medicine, vol. 366, no. 21, pp. 1968-1977, May 2012, doi: 10.1056/NEJMOA1113354/SUPPL_FILE/NEJMOA1113354_DISCLOSURES.PDF.
[14]C. Combet, C. Blanchet, C. Geourjon, and G. Delsage, “NPS@: Network Protein Sequence Analysis,” Trends Biochem Sci, vol. 25, no. 3, pp. 147-150, March 2000, doi: 10.1016/50968-0004(99)01540-6.
[15]A. Bateman et al., “UniProt: the universal protein knowledgebase,” Nucleic Acids Res, vol. 45, no. D1, pp. D158-D169, January 2017, doi: 10.1093/NAR/GKW1099.
[16]M. L. Gefter, D. H. Margulies, and M. D. Scharff, “A simple method for polyethylene glycol-promoted hybridization of mouse myeloma cells,” Somatic Cell Genet, vol. 3, no. 2, pp. 231-236, March 1977, doi: 10.1007/BF01551818.
[17]J. M. B. Sand, G. Martinez, A. K. Midjord, M. A. Karsdal, D. J. Leeming, and P. Lange, “Characterization of serological neo-epitope biomarkers reflecting collagen remodelling in clinically stable chronic obstructive pulmonary disease,” Clin Biochem, vol. 49, no. 15, pp. 1144-1151, October 2016, doi: 10.1016/J.CLINBIOCHEM.2016.09.003.
[18]T. S. Prior et al., “Clusters of comorbidities in idiopathic pulmonary fibrosis,” Respir Med, vol. 185, August 2021, doi: 10.1016/J.RMED.2021.106490.
[19] Chakraborty A, Mastalerz M, Ansari M, Schiller H B, Staab-Weijnitz C A. Emerging Roles of Airway Epithelial Cells in Idiopathic Pulmonary Fibrosis. Cells. 2022; 11(6):1050. https://doi.org/10.3390/cells11061050
[20] Rajagopal, K., Bryant, A. J., Sahay, S., Wareing, N., Zhou, Y., Pandit, L. M., & Karmouty-Quintana, H. (2021). Idiopathic pulmonary fibrosis and pulmonary hypertension: Heracles meets the Hydra. British journal of pharmacology, 178(1), 172-186. https://doi.org/10.1111/bph.15036
[21] King C S, Shlobin O A. The Trouble With Group 3 Pulmonary Hypertension in Interstitial Lung Disease: Dilemmas in Diagnosis and the Conundrum of Treatment. Chest. 2020; 158(4):1651-1664. doi:10.1016/j.chest.2020.04.046

1. A method of immunoassay, said method comprising;
i) contacting a patient sample with a monoclonal antibody that specifically binds to the N-terminus amino acid sequence VDTLPQKPRE (SEQ ID NO: 1); and
ii) detecting and determining the amount of binding between said monoclonal antibody and peptides in the sample.

2. The method as claimed in claim 1, wherein the method is a method of immunoassay for detecting and/or monitoring chronic obstructive pulmonary disease or idiopathic pulmonary fibrosis or a particular level of severity thereof in a patient, the method further comprising;
iii) correlating said amount of binding with values associated with normal healthy subjects and/or values associated with known disease severity and/or values obtained from said patient at a previous time point and/or with a predetermined cut-off value.

3. The method as claimed in claim 2, wherein the method is a method for detecting and/or monitoring chronic obstructive pulmonary disease or a particular level of severity thereof in a patient.

4. The method as claimed in claim 2, wherein the method is a method for detecting and/or monitoring a particular level of severity of idiopathic pulmonary fibrosis in a patient; or wherein the method is a method for detecting and/or monitoring idiopathic pulmonary fibrosis with pulmonary hypertension in a patient.

5. The method as claimed in claim 1, wherein the patient sample is selected from blood, plasma or serum.

6. The method as claimed in claim 1, wherein the monoclonal antibody does not specifically bind to a peptide having the N-terminus amino acid sequence LVDTLPQKPRE (SEQ ID NO: 3).

7. The method as claimed in claim 1, wherein the monoclonal antibody does not specifically bind to a peptide having the N-terminus amino acid sequence DTLPQKPRE (SEQ ID NO: 4).

8. The method as claimed in claim 1, wherein the monoclonal antibody is raised against a synthetic peptide having the N-terminus amino acid sequence VDTLPQKPRE (SEQ ID NO: 1).

9. The method as claimed in claim 1, wherein the immunoassay is a competition assay or a sandwich assay.

10. The method as claimed in claim 1, wherein the immunoassay is a radio-immunoassay or an enzyme-linked immunosorbent assay.

11. A monoclonal antibody that specifically binds to the N-terminus amino acid sequence VDTLPQKPRE (SEQ ID NO: 1).

12. The monoclonal antibody as claimed in claim 11, wherein the monoclonal antibody does not specifically bind to a peptide having the N-terminus amino acid sequence LVDTLPQKPRE (SEQ ID NO: 3).

13. The monoclonal antibody as claimed in claim 11, wherein the monoclonal antibody does not specifically bind to a peptide having the N-terminus amino acid sequence DTLPQKPRE (SEQ ID NO: 4).

14. The monoclonal antibody as claimed in claim 11, wherein the monoclonal antibody is raised against a synthetic peptide having the N-terminus amino acid sequence VDTLPQKPRE (SEQ ID NO: 1).

15. An immunoassay kit comprising the monoclonal antibody as claimed in claim 11, and at least one of:
a streptavidin coated well plate;
a biotinylated peptide VDTLPQKPRE-L-Biotin (SEQ ID NO: 17), wherein L is an optional linker;
a secondary antibody for use in a sandwich immunoassay;
a calibrator protein comprising the N-terminus amino acid sequence VDTLPQKPRE (SEQ ID NO: 1);
an antibody biotinylation kit;
an antibody HRP labelling kit; or
an antibody radiolabelling kit.


SEQUENCE LISTING (28 sequences)

SEQ ID NO: 1 - protein, 10 aa, synthetic construct
VDTLPQKPRE

SEQ ID NO: 2 - protein, 10 aa, synthetic construct
AMELFSPDQD

SEQ ID NO: 3 - protein, 11 aa, synthetic construct
LVDTLPQKPRE

SEQ ID NO: 4 - protein, 9 aa, synthetic construct
DTLPQKPRE

SEQ ID NO: 5 - protein, 11 aa, synthetic construct
AAMELFSPDQD

SEQ ID NO: 6 - protein, 9 aa, synthetic construct
MELFSPDQD

SEQ ID NO: 7 - protein, 17 aa, Mus musculus
KSSQSLFNSGTQKNYLA

SEQ ID NO: 8 - protein, 7 aa, Mus musculus
WASTRES

SEQ ID NO: 9 - protein, 8 aa, Mus musculus
KQSYNLLT

SEQ ID NO: 10 - protein, 5 aa, Mus musculus
DYSMH

SEQ ID NO: 11 - protein, 17 aa, Mus musculus
WINTETGEPTYADDFKG

SEQ ID NO: 12 - protein, 8 aa, Mus musculus
MITVAMDY

SEQ ID NO: 13 - protein, 79 aa, Mus musculus
KSSQSLFNSGTQKNYLAWYQQKPGQSPKLLIYWASTRESGVPDRFTGSGSGTDFALTITSVQAEDLAVYYCKQSYNLLT

SEQ ID NO: 14 - protein, 76 aa, Mus musculus
DYSMHWVKQAPGKGLKWVGWINTETGEPTYADDFKGRFAFSLETSASTAYLRVNNLKNEDTATYFCGTMITVAMDY

SEQ ID NO: 15 - protein, 111 aa, Mus musculus
DIVLSQSPSSLAVSAGEKVTMSCKSSQSLFNSGTQKNYLAWYQQKPGQSPKLLIYWASTRESGVPDRFTGSGSGTDFALTITSVQAEDLAVYYCKQSYNLLTFGAGTKLEL

SEQ ID NO: 16 - protein, 117 aa, Mus musculus
QIQLVQSGPELKKPGETVKISCKASGYTFTDYSMHWVKQAPGKGLKWVGWINTETGEPTYADDFKGRFAFSLETSASTAYLRVNNLKNEDTATYFCGTMITVAMDYWGQGTSVTVSS

SEQ ID NO: 17 - protein, 10 aa, synthetic construct
  MOD_RES 10: note = biotinylated with optional linker
VDTLPQKPRE

SEQ ID NO: 18 - protein, 10 aa, synthetic construct
  MOD_RES 10: note = biotinylated with optional linker
AMELFSPDQD

SEQ ID NO: 19 - protein, 13 aa, synthetic construct
  MOD_RES 13: note = linked to keyhole limpet hemocyanin
VDTLPQKPREGGC

SEQ ID NO: 20 - protein, 11 aa, synthetic construct
  MOD_RES 11: note = biotinylated
VDTLPQKPREK

SEQ ID NO: 21 - protein, 10 aa, synthetic construct
QMAGLDEKSG

SEQ ID NO: 22 - protein, 10 aa, synthetic construct
QAAFSQYKKV

SEQ ID NO: 23 - protein, 10 aa, synthetic construct
  MOD_RES 1: note = biotinylated
QAAFSQYKKV

SEQ ID NO: 24 - protein, 472 aa, Mus musculus
MAWVWTLLFLMAAAQSIQAQIQLVQSGPELKKPGETVKISCKASGYTFTDYSMHWVKQAPGKGLKWVGWINTETGEPTYADDFKGRFAFSLETSASTAYLRVNNLKNEDTATYFCGTMITVAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 25 - protein, 239 aa, Mus musculus
MDSQAQVLILLLLWVSGTCGDIVLSQSPSSLAVSAGEKVTMSCKSSQSLFNSGTQKNYLAWYQQKPGQSPKLLIYWASTRESGVPDRFTGSGSGTDFALTITSVQAEDLAVYYCKQSYNLLTFGAGTKLELRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 26 - protein, 31 aa, Homo sapiens
AQLKKLVDTLPQKPRESIIKLMEKIAQSSLC

SEQ ID NO: 27 - protein, 31 aa, Rattus rattus
TQLKRLVDTLPQETRINIVKLTEKILTSPLC

SEQ ID NO: 28 - protein, 31 aa, Mus musculus
TQLKRLVDTLPQETRINIMKLTEKILTSPLC